(19) United States
(12) Patent Application Publication
(10) Pub. No.: US 2016/0045340 A1
(43) Pub. Date: Feb. 18, 2016

(54) Radial Volume Adjustment Device

(71) Applicant: Board of Regents, The University of Texas System, Austin, TX (US)

(72) Inventors: Meagan Vaughan, Ovilla, TX (US); Richard Crawford, Austin, TX (US); Gordon Bosker, San Antonio, TX (US)

(21) Appl. No.: 14/779,223

(22) PCT Filed: Mar. 27, 2014
(86) PCT No.: PCT/US2014/032015

§ 371 Date: Sep. 22, 2015

Related U.S. Application Data
(60) Provisional application No. 61/805,811, filed on Mar. 27, 2013.

(51) Int. Cl. A61F 2/80

(52) U.S. Cl. CPC A61F 2/80

(57) ABSTRACT

The present disclosure relates to a radial volume adjustment device. The device includes a connection plate. The device further includes a socket wall comprising a plurality of socket wall components, at least one of the plurality of socket wall components comprising a channel, the channel forming an arcuate path curving towards a center of the connection plate. The device further includes at least one attachment member configured to couple with the connection plate and pass through the at least one channel such that the at least one socket wall component may move in a path defined by the at least one attachment member and the channel. The movement of the at least one attachment member causes a volume defined by the socket wall to change.

### TECHNICAL FIELD

The present disclosure relates generally to prosthetic devices, and more particularly, to a radial volume adjustment device.

### BACKGROUND

Many people each year may lose a limb due to any of a variety of causes, including accident, military service, medical necessity, osteosarcoma, diabetes, or any other of a variety of reasons. This may be a particular problem in areas of the world in which advanced medical care may not be readily available.

For individuals with a lost limb, a prosthetic limb may be utilized to compensate for the loss of the limb. For example, a prosthetic leg may be used both to facilitate walking and for aesthetic reasons. Prosthetics may have a socket to interface with the residual limb of a patient.

Residual limb volume may vary for patients with prosthetic limbs. For example, some studies suggest that limb volume may change between −11% and 7% in a single day due to changing activity level and weight. Volume changes of only 3% to 5% may cause a patient to have difficulty in attaching the prosthetic socket. Some volume and/or size changes to a residual limb may be caused by normal growth in children. For example, a tibia may change on average 22.02 millimeters (mm) per year for boys and 19.81 mm per year for girls between the ages of ten and seventeen. Additionally, between the ages of two and ten, an average girl gains 3.65 kilograms (kg) per year and an average boy gains 3.25 kg per year. For many young patients, this may mean that a new prosthetic is required every year at least until the age of five. Thus, there is a need in the art for an improved prosthetic socket.

### SUMMARY

In accordance with some embodiments of the present disclosure, a radial volume adjustment device is disclosed. The socket includes a spacer plate with one or more channels forming an arcuate path curving towards a center of the adjustment spacer. The socket further includes an adjustment spacer consisting of a plurality of sections. The socket further includes at least one attachment member configured to couple with the spacer plate and pass through the at least one channel such that the at least one adjustment spacer section may move in a path defined by the at least one attachment member and the at least one channel. The movement of the at least one attachment member causes the sections to move inwardly and outwardly in the radial direction relative to the spacer plate. The socket further includes a plurality of socket wall components coupled to the adjustment spacer sections. The adjustment of the adjustment spacer sections causes a volume defined by the plurality of socket wall components to change.

In accordance with another embodiment of the present disclosure, a radial volume adjustment device is disclosed. The device includes a connection plate. The device further includes a socket wall comprising a plurality of socket wall components, at least one of the plurality of socket wall components comprising a channel, the channel forming an arcuate path curving towards a center of the connection plate. The device further includes at least one attachment member configured to couple with the connection plate and pass through the at least one channel such that the at least one socket wall component may move in a path defined by the at least one attachment member and the channel. The movement of the at least one attachment member causes a volume defined by the socket wall to change.

In accordance with a further embodiment of the present disclosure, a method of manufacturing a radial volume adjustment device is disclosed. The method includes creating a mold of a residual limb. The method further includes crafting a plurality of socket wall components using the mold of the residual limb. The method further includes inserting a channel in the plurality of socket wall components. The channel forms an arcuate path curving towards the center of the socket base. The method further includes connecting the plurality of socket wall components to a socket base to form a radial volume adjustment device, wherein the movement of the at least one socket wall component causes a volume defined by the plurality of socket wall components to change.

### BRIEF DESCRIPTION OF THE DRAWINGS

A more complete understanding of the disclosed embodiments and advantages thereof may be acquired by referring to the following description taken in conjunction with the accompanying drawings, in which like reference numbers indicate like features, and wherein:

FIG. 1 illustrates an example prosthetic, including a socket, in accordance with the teachings of present disclosure;

FIG. 2 illustrates an isometric view of an example embodiment of a socket base, in accordance with the teachings of present disclosure;

FIG. 3 illustrates a side view of an example embodiment of a socket base, in accordance with the teachings of present disclosure;

FIG. 4 illustrates an example embodiment of an adjustment spacer, in accordance with the teachings of the present disclosure;

FIG. 5 illustrates an example embodiment of a spacer plate, in accordance with the teachings of the present disclosure;

FIGS. 6A-6F illustrate an example embodiment of a socket base adjusting its spacing, in accordance with the teachings of the present disclosure;

FIG. 7 illustrates an example embodiment of a prosthetic socket, in accordance with the teachings of the present disclosure;

FIGS. 8 and 9 illustrate an alternative example embodiment of a prosthetic socket, in accordance with the teachings of the present disclosure;

FIGS. 10 and 10A illustrates an alternative example embodiment of a socket wall, in accordance with the teachings of the present disclosure;

FIGS. 11 and 11A illustrates an alternative example embodiment of a socket base, in accordance with the teachings of the present disclosure;

FIGS. 12A-12C illustrate an alternative example embodiment of a socket base, in accordance with the teachings of the present disclosure;

FIGS. 13A-13D illustrate embodiments of various attachment supports, in accordance with the teachings of the present disclosure; and

FIG. 14 illustrates another alternative example embodiment of a socket base, in accordance with the teachings of the present disclosure.

### DETAILED DESCRIPTION

The present disclosure relates to an adjustable socket for a prosthetic limb. A socket base comprising a spacing plate and an adjustment spacer may be configured to rotate relative to each other, while remaining substantially parallel. This rotation may cause an internal volume of the socket to vary by causing a socket wall coupled with the socket base to change its radial position relative to the center of the socket.

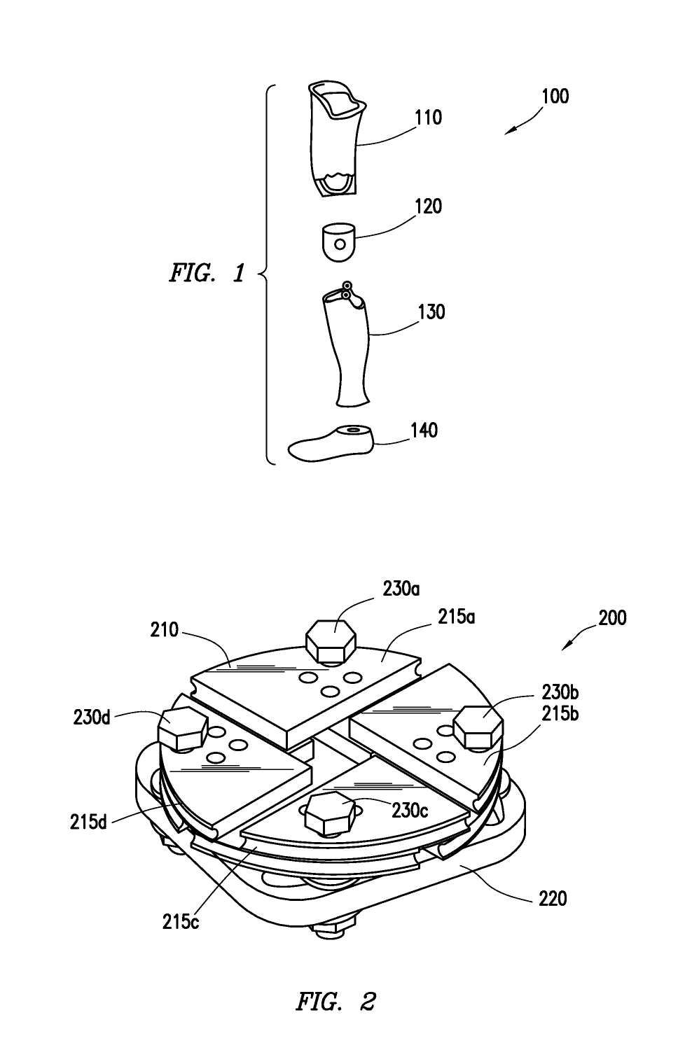

FIG. 1 illustrates an example of a prosthetic leg 100, in accordance with the teachings of the present disclosure. As shown in FIG. 1, prosthetic leg 100 may include a variety of components, including socket 110, knee 120, shank 130, and foot-ankle 140. Socket 110 may be configured to interface with a residual limb of a patient. Prosthetic leg 100 may include additional components or may not include all components shown in the example embodiment illustrated in FIG. 1.

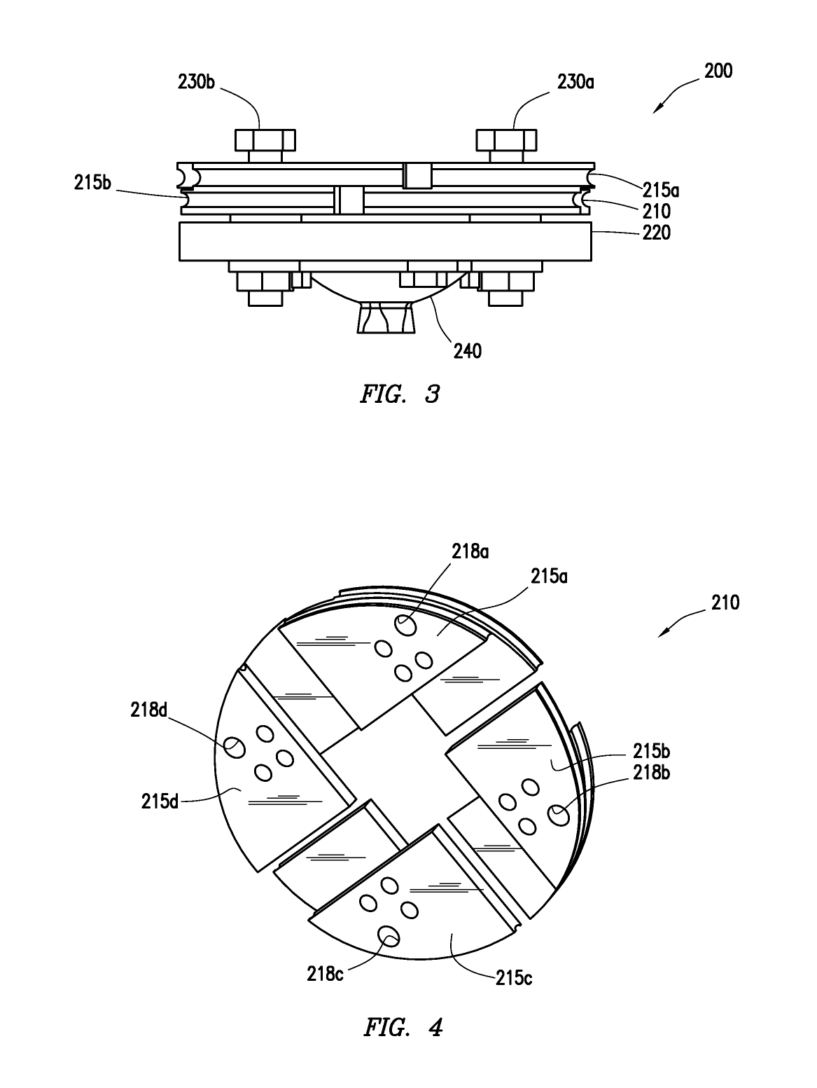

FIG. 2 illustrates an isometric view of an example embodiment of socket base 200, in accordance with the teachings of the present disclosure. FIG. 3 illustrates a side view of an example embodiment of socket base 200, in accordance with the teachings of the present disclosure. Socket base 200 may include adjustment spacer 210 and spacer plate 220. Adjustment spacer 210 may include a plurality of individual wings 215 (for example, individual wings 215a-215d) arranged to form adjustment spacer 210. Socket base 200 may additionally include releasable attachment members 230, for example, releasable attachment members 230a-230d. Socket base 200 may additionally include an engaging member 240, as shown in FIG. 3, configured to engage socket base 200 with an additional prosthetic component, for example a component of prosthetic leg 100 as shown in FIG. 1.

Adjustment spacer 210 may be made of any suitable material of sufficient strength to support the weight of a patient and any forces that may be generated by utilization of the prosthetic. For example, adjustment spacer 210 may be made of steel, stainless steel, titanium, hard plastics, or any other suitable material. Attachment member 230 may include a nut and bolt, screw, peg, or any other suitable mechanical component configured to couple spacer plate 210 and adjustment spacer 220 while allowing movement of individual wings 215 relative to each other. Spacer plate 220 may be made of any suitable material of sufficient strength to support the weight of a patient and any forces that may be generated by utilization of the prosthetic. For example, spacer plate 220 may be made of steel, stainless steel, titanium, hard plastics, or any other suitable material.

Individual wings 215 of adjustment spacer 210 may be attached, coupled, or joined in any of a variety of ways such that individual wings 215 may move relative to each other in a coordinate plane substantially parallel with the face of adjustment spacer 210, without substantially moving from that plane. Alternatively, in some embodiments, individual wings 215 may not be coupled in any way, and may merely overlap one another to form adjustment spacer 210. For example, as shown in FIG. 4, individual wings 215a-215d may have overlapping portions that are not attached. In some embodiments adjustment spacer 210 may additionally include regions 218 suitable for receiving attachment members 230. In some embodiments, each individual wing 215a-215d may have a respective region 218a-218d for receiving attachment members 230a-230d. Further, regions 218a-218d and attachment members 230a-230d may be configured to prevent movement of each individual wing 215a-215d relative to its respective attachment member 230a-230d. Adjustment spacer 210 is shown in FIG. 4 as having four individual wings 215a-215d, however adjustment spacer 210 may have fewer than or more than four individual wings 215.

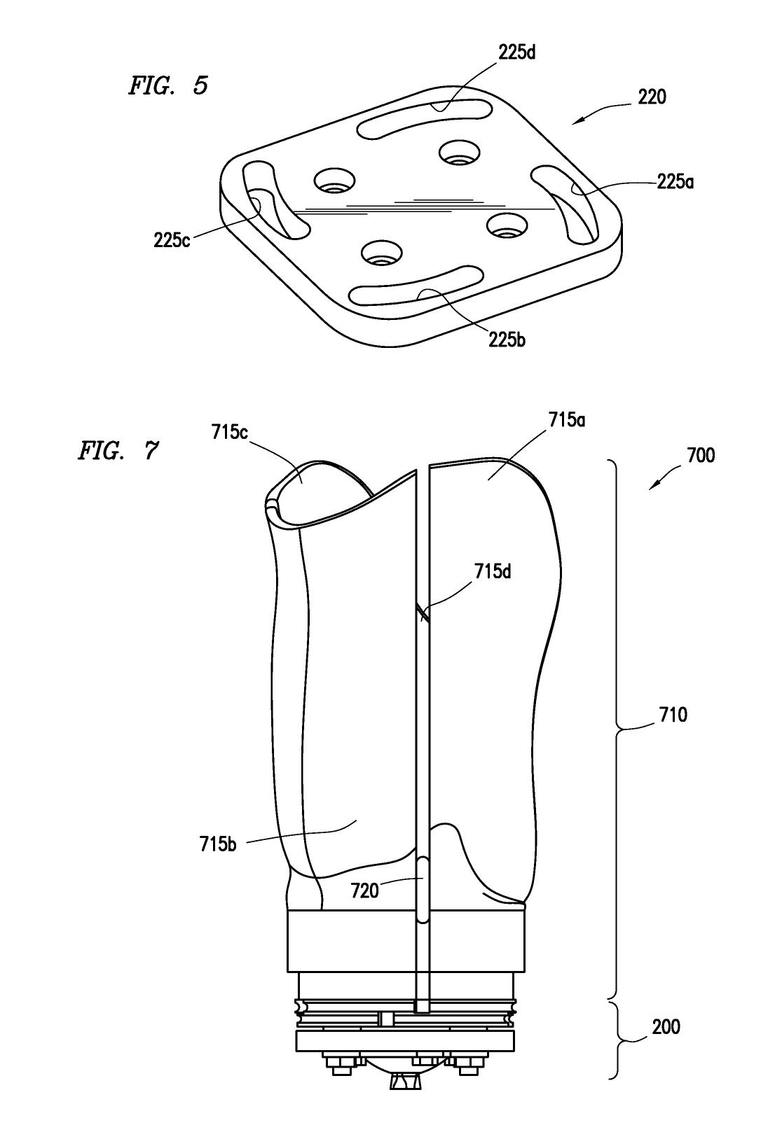

FIG. 5 illustrates an example embodiment of spacer plate 220, in accordance with the teachings of the present disclosure. As shown in FIG. 5, spacer plate 220 may include channels 225 configured to receive attachment members 230 (for example, channels 225a-225d corresponding to respective attachment members 230a-230d as shown in FIG. 2). Further, channels 225 may be configured to allow controlled movement of spacer plate 220 and adjustment spacer 210, as shown in FIG. 2, relative to each other. For example, channels 225 may act as guides for attachment members 230 such that each respective region 218 for receiving attachment member 230 follows the respective channel 225 that receives the respective attachment member 230. Channels 225 may follow an arcuate path curving slightly in towards the middle of spacer plate 220.

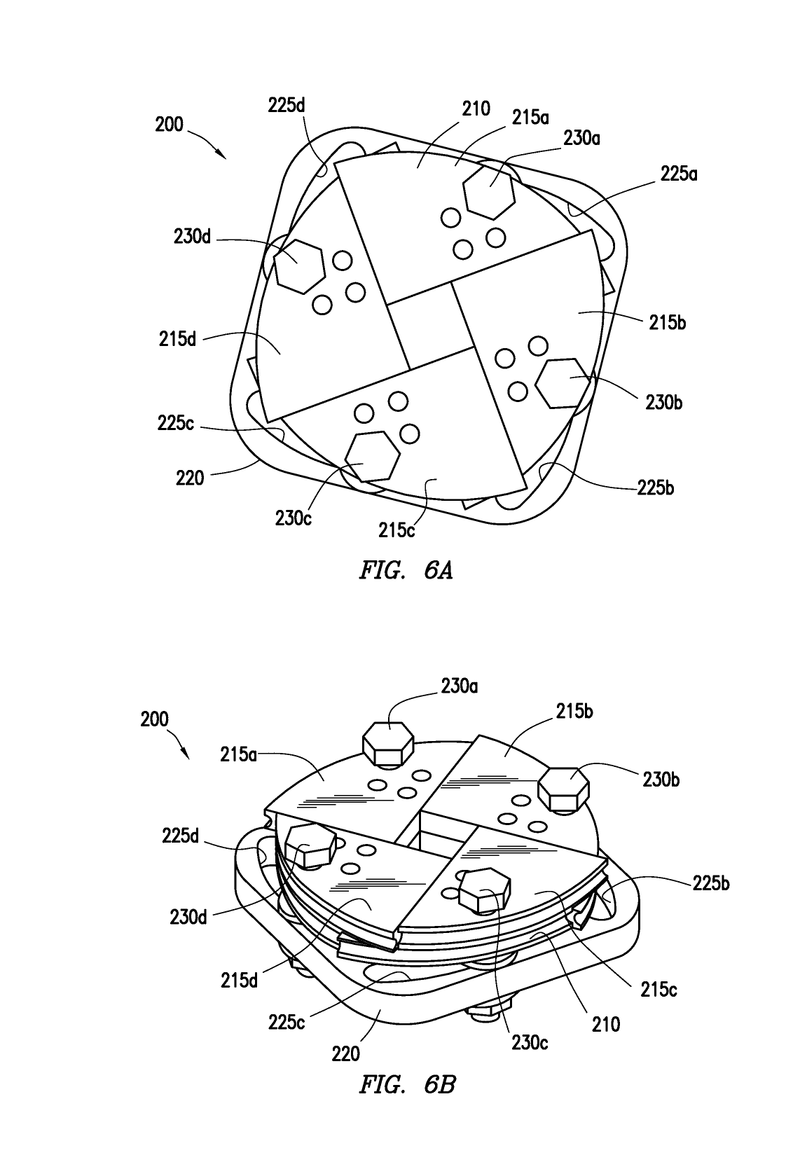

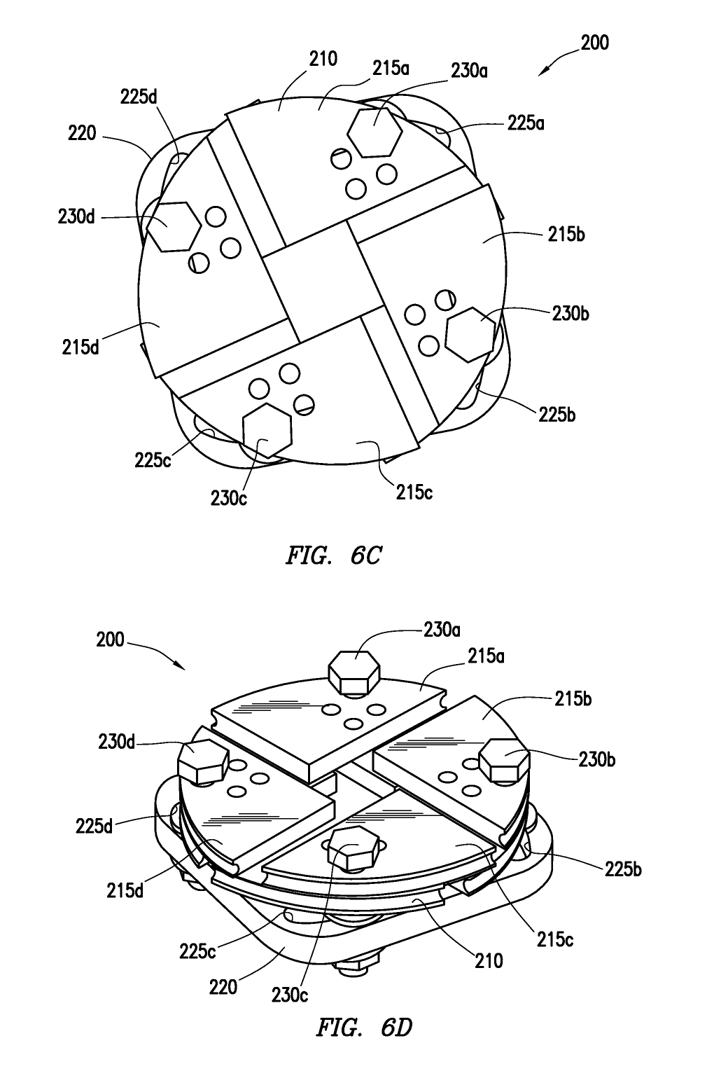

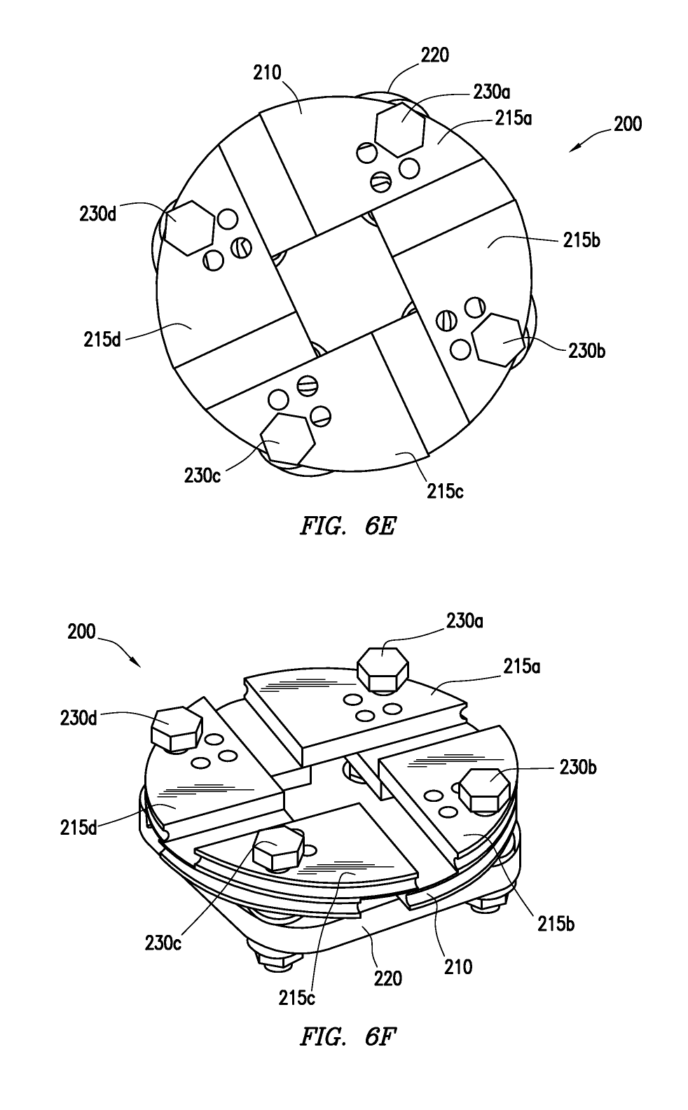

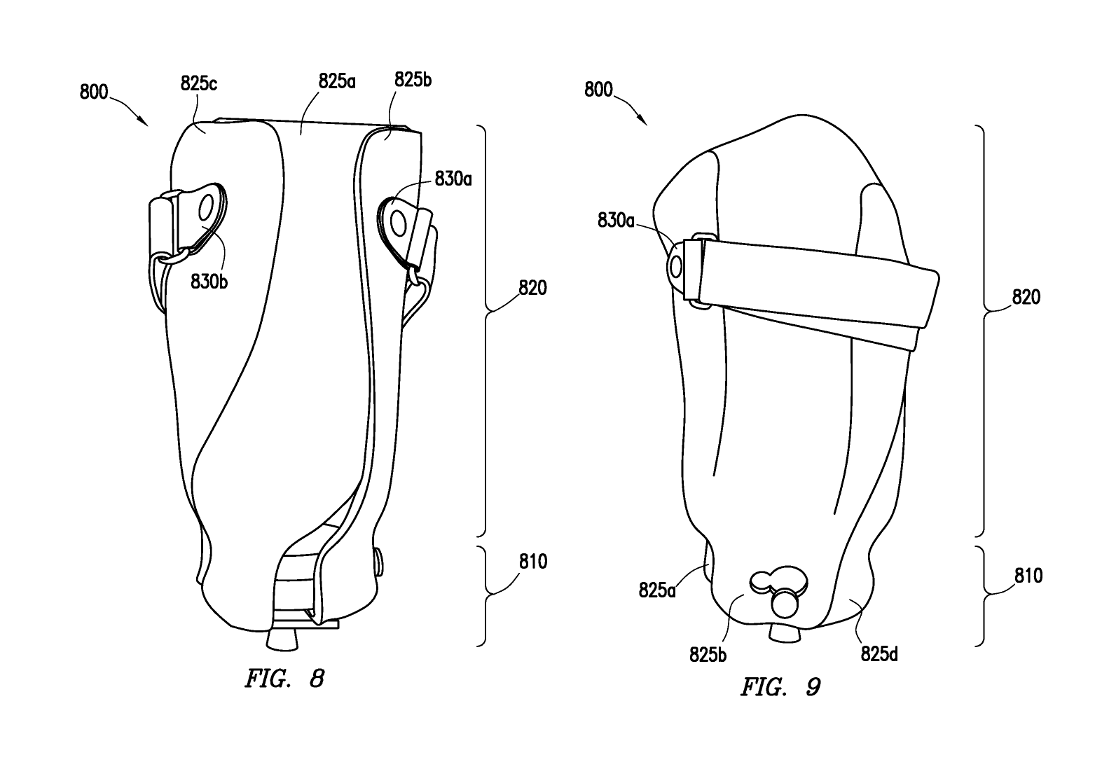

FIGS. 6A-6F illustrate an example embodiment of socket base 200 adjusting its spacing, in accordance with teachings of the present disclosure. As shown in FIGS. 6A-6F, adjustment spacer 210 and spacer plate 220 may rotate relative to each other and by so doing, may cause adjustment spacer 210 to expand. FIGS. 6A and 6B show the same instance in time, but a different perspective view. The same is true for FIGS. 6C and 6D as well as FIGS. 6E and 6F. FIGS. 6A and 6B illustrate a first contracted position. Attachment members 230 are at the end of channels 225 closest to the middle of spacer plate 220. As can be seen in FIGS. 6C and 6D, as attachment members 230 move along channels 225, individual wings 215 move relative to each other causing adjustment spacer 210 to expand. FIGS. 6C and 6D show adjustment spacer 210 partially expanded. As can be seen in FIGS. 6E and 6F, when attachment members 230 are at the end of channels 225, adjustment spacer 210 is at a maximally expanded position.

Described in an alternative way, adjustment spacer 210 and spacer plate 220 may be rotated relative to each other such that the faces of the two components remain substantially parallel. While rotating relative to each other, attachment members 230 and channels 225 may cause individual wings 215 to extend from or contract towards the center of adjustment spacer 210 while moving radially relative to spacer plate 220.

In some embodiments, attachment members 230 may be loosened before adjustment spacer 210 and spacer plate 220 are rotated relative to each other. Once the desired degree of expansion of adjustment spacer 210 is reached, attachment members 230 may be tightened to prevent rotation of adjustment spacer 210 and spacer plate 220 relative to each other.

FIG. 7 illustrates an example embodiment of prosthetic socket 700, in accordance with the teachings of the present disclosure. As shown in FIG. 7, prosthetic socket 700 may include socket base 200. Prosthetic socket 700 may additionally include a socket wall 710 comprising a plurality of socket wall components 715 (for example, socket wall components 715a-715d). Prosthetic socket 700 may additionally include pad 720. By way of example and not limitation, pad 720 may be a gel insert.

Socket wall 710 may be made of any of a variety of materials. The material should be of sufficient strength to support attachment of prosthetic socket 700 to a patient's residual limb, but should preferably be light and at least slightly flexible. For example, a sturdy plastic or composite material may be used. Additionally, carbon fibers with some sort of binding agent or laminating agent may be used.

In some embodiments, socket wall components 715 may be coupled with individual wings 215 such that when a particular individual wing 215 moves, the respective socket wall component 715 also moves. Socket wall components 715 may overlap one another with sufficient overlap such that as individual wings 215 are moved, a gap is not created in a majority of socket wall 710 as socket wall components 715 slide past each other. For example, a gap may occur below pad 720 but overlap may prevent a gap from occurring at and above pad 720 in socket wall 710. In some embodiments, a gap is prevented from all possible positions of individual wings 215, that is, socket wall components 715 overlap from the most contracted position of adjustment spacer 210 to the most expanded position of adjustment spacer 210.

As can be appreciated, as adjustment spacer 210 is rotated relative to spacer plate 220, the volume enclosed by socket wall 710 may be varied with the corresponding radial expansion of adjustment spacer 210. This variation in volume may allow for a more comfortable and more appropriate fit for a patient with respective changes in volume to their residual limb.

As an illustrative example, and in no way limiting, a vertical height of adjustment spacer 210 and spacer plate 220 may be one inch and spacer plate 220 may be four and a half inches across. From the most contracted position to the most expanded position, adjustment spacer 210 may experience a 0.4 inch radial expansion. This may correspond to a total volume change of approximately 30%, or in other words, if starting from the middle position (as shown in FIGS. 6C and 6D), there may be a +/−15% volume change (to the position shown in FIGS. 6A and 6B or to the position shown in FIGS. 6E and 6F). It will be appreciated that the change may be larger or smaller by varying the size, length, or radius of curvature of channels 225. This may increase the bulk of socket base 200.

FIGS. 8 and 9 illustrate an alternative example embodiment of prosthetic socket 800. Prosthetic socket 800 may include socket base region 810, and socket wall 820 including a plurality of socket wall components 825 (for example, socket wall components 825a-825d), and attachment supports 830 (for example, attachment supports 830a and 830b). Prosthetic socket 800 may be configured to engage with a pin or other component attached to the residual limb of a patient, for example, by stockings or hose with a pin at the end. Prosthetic socket 800 may further be configured to lock the pin into place when engaged with prosthetic socket 800.

As described with respect to prosthetic socket 700 as discussed with respect to FIG. 7, socket wall components 825 may overlap each other. For example, socket wall components 825 may overlap one another with sufficient overlap such that as socket wall components 825 slide past each other, no gap is created through the majority of socket wall 820. For example, a gap may occur in the bottom one third or one fourth of socket wall 820, or less, but overlap may prevent a gap from occurring in the remainder of socket wall 820. In some embodiments, a gap is prevented in the majority of socket wall 820 from all possible radial expansions or contractions of socket wall 820. In some embodiments, socket wall 820 may comprise four or more socket wall components 825. In other embodiments, socket wall 820 may comprise two or three socket wall components 825. Socket wall components 825 may be manufactured of both rigid materials and flexible materials. Socket wall components 825 may be arranged to form socket wall 820 in a manner alternating between a rigid socket wall component 825 and a flexible socket wall component 825.

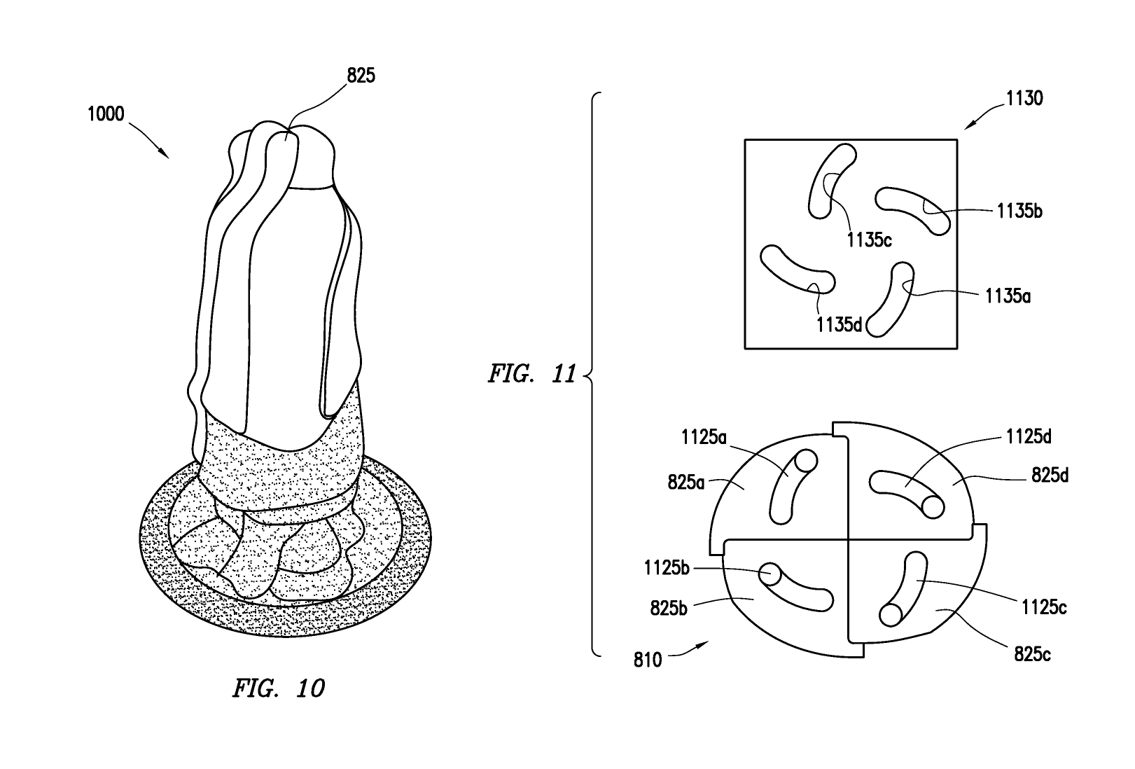

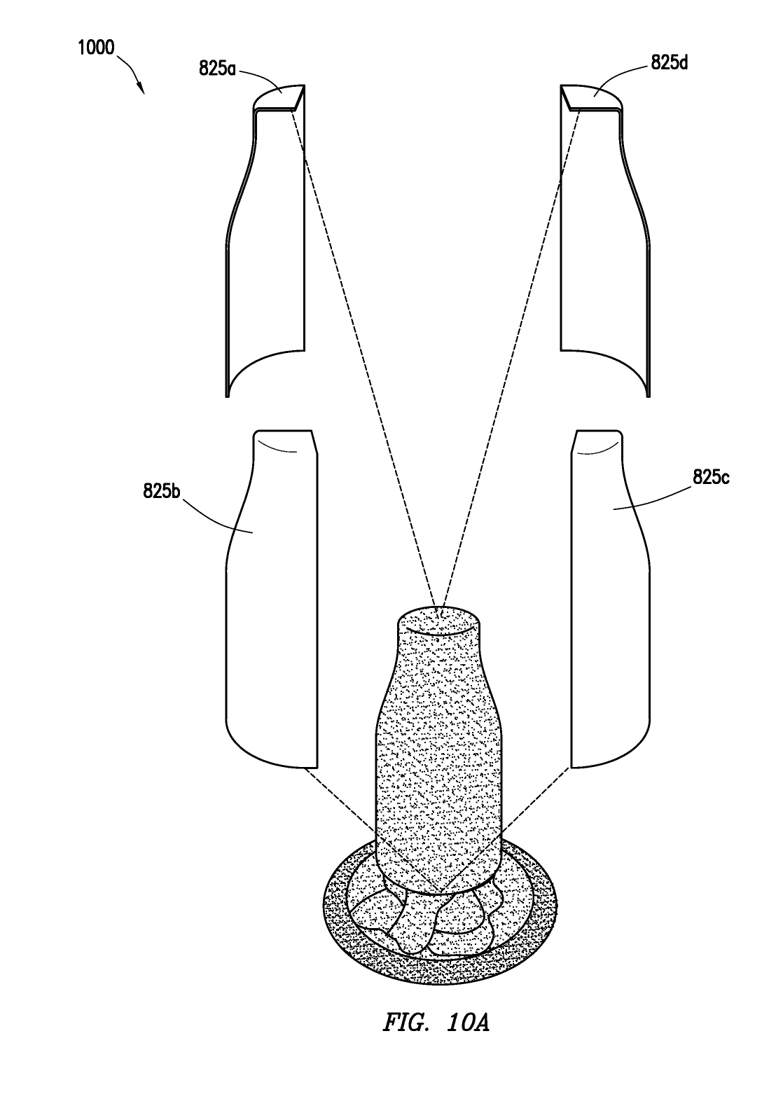

In some embodiments, to form socket wall components 825, a mold of a patient's residual limb may be created. The mold may be used as a model for crafting socket wall components 825. For example, as shown in FIG. 10, a first set of one or more socket wall components 825 may be crafted. As shown in FIG. 10A, socket wall components 825a-825d may be created through the use of the mold shown in FIG. 10. In addition to the model of the patient's residual limb, other place holders may be used to reserve space for overlap with other socket wall components 825. Following the crafting of the first set of one or more socket wall components 825, a second set of one or more socket wall components 825 may be crafted. Any number of iterations may be utilized in crafting the socket wall components 825. In some embodiments, some techniques for crafting socket wall components 825 may allow for creation of all socket wall components 825 in a single iteration. Socket wall components 825 may be crafted using carbon fiber lamination manufacturing techniques.

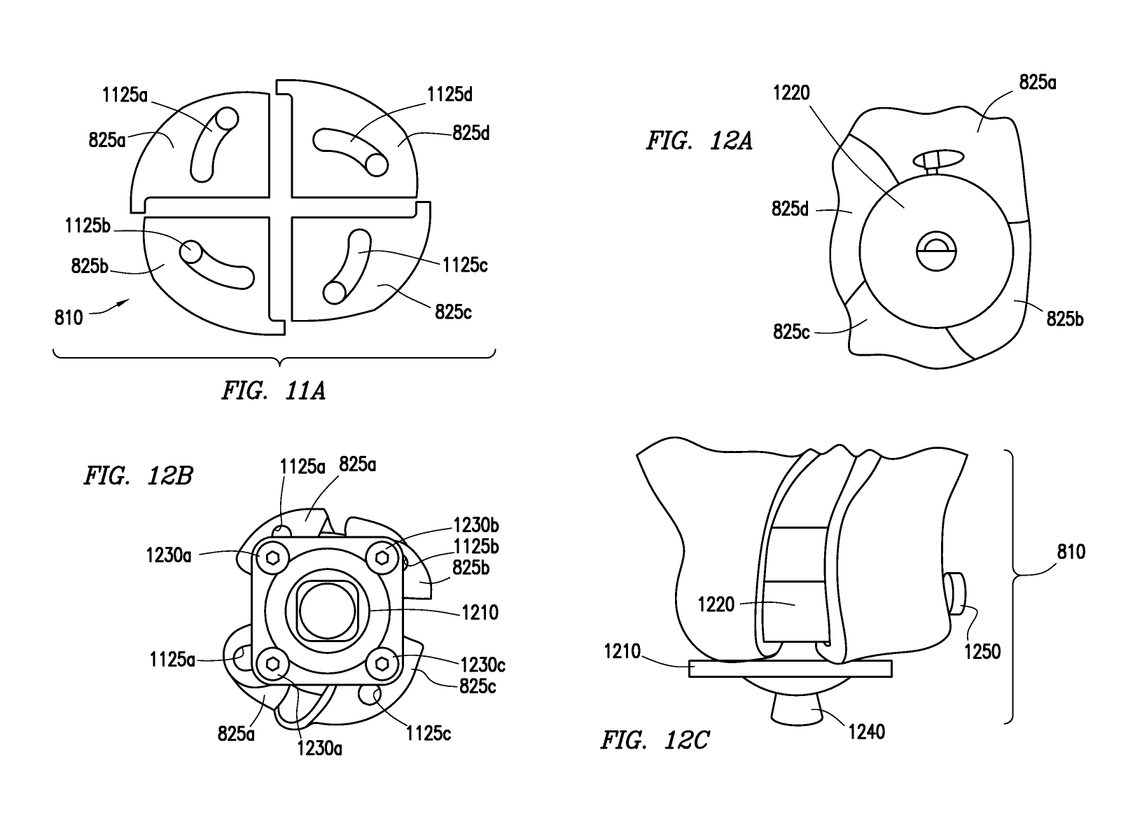

FIG. 11 illustrates an alternative example embodiment of socket base 810, in accordance with the teachings of the present disclosure. As shown in FIG. 11, socket base 810 may include channels 1125 (for example, channels 1125a-1125d) as part of socket wall components 825. In this way, rather than having socket wall components 715 as shown in FIG. 7 that are merely coupled to the individual wings 215 of adjustment spacer 210 as shown in FIG. 2, socket wall component 825 may be a unitary body with components of socket base 810, for example with individual wings 215. Socket base 810 may be made of more than one socket wall component 825a-825d, as shown in FIG. 11A. To insert channels 1125, a manufacturer or designer may use a template 1130 that includes channels 1135 (for example, channels 1135a-1135d) such that the desired degree of rotation equates to the proper amount of radial contraction or expansion. The socket wall components may then be connected to socket base 810.

FIGS. 12A-12C illustrate an alternative example embodiment of socket base 810, in accordance with the teachings of the present disclosure. As shown in FIGS. 12A-12C, other components of socket base 810 may also be included. For example, socket base 810 may include connection plate 1210 comprising engaging member 1240, base component 1220, attachment members 1230, and releasing member 1250. For example, base component 1220 may be configured to engage with a pin coupled to a patient's residual limb, and releasing member 1250 may be configured to release the pin from engagement with base component 1220. Further, connection plate 1210 may be removably attached to base component 1220 via attachment members 1230 (for example, attachment members 1230a-1230d). Socket 800 may be coupled to additional prosthetic components (not shown) via engaging member 1240.

The internal volume defined by socket wall 810 may be modified using channels 1125, attachment members 1230, and connection plate 1210. For example, by loosening attachment member 1230a, corresponding socket wall component 825a may be rotated such that engaging member 1230a remains in channel 1125a. As this occurs, socket wall component 825a slides by socket wall components 825b and 825d. Because of the arcuate path of channel 1125a, not only does socket wall component 825a move around the center of connection plate 1210, but it also moves in towards or away from the center of connection plate 1210. By moving socket wall component 825a along the path defined by channel 1125a and attachment member 1230a, and corresponding motion by socket wall components 825b-825d, the volume defined by socket wall 820 may be modified. In this way, the volume may be modified in a comparable manner to that observed with respect to the movement of individual wings 215 with socket wall components 715 coupled thereto. For example, a comparable volume difference of approximately 30% may be observed by moving socket wall components 825 from one end of channels 1125 to the other.

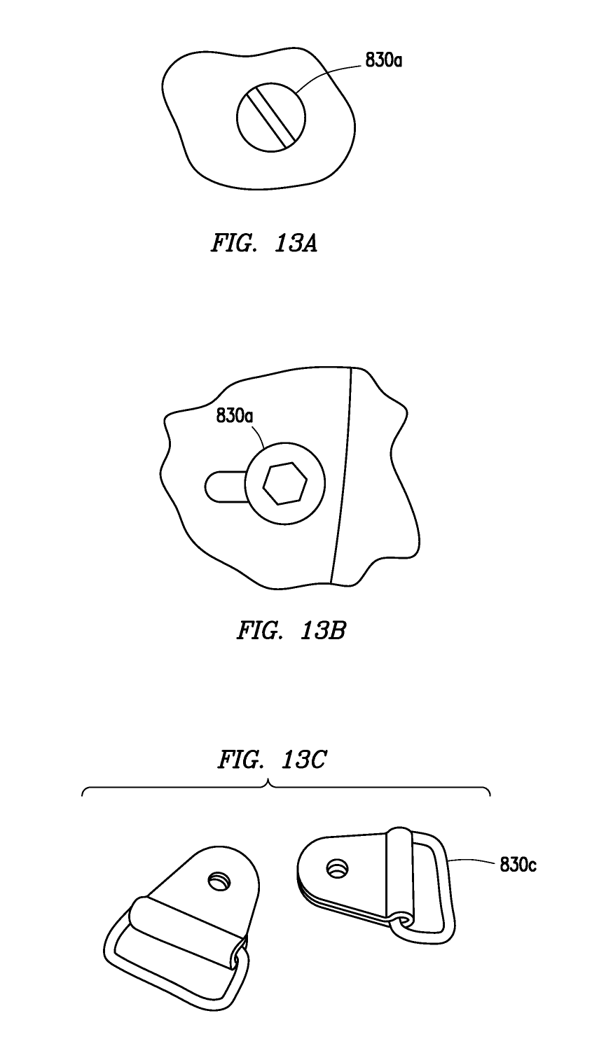

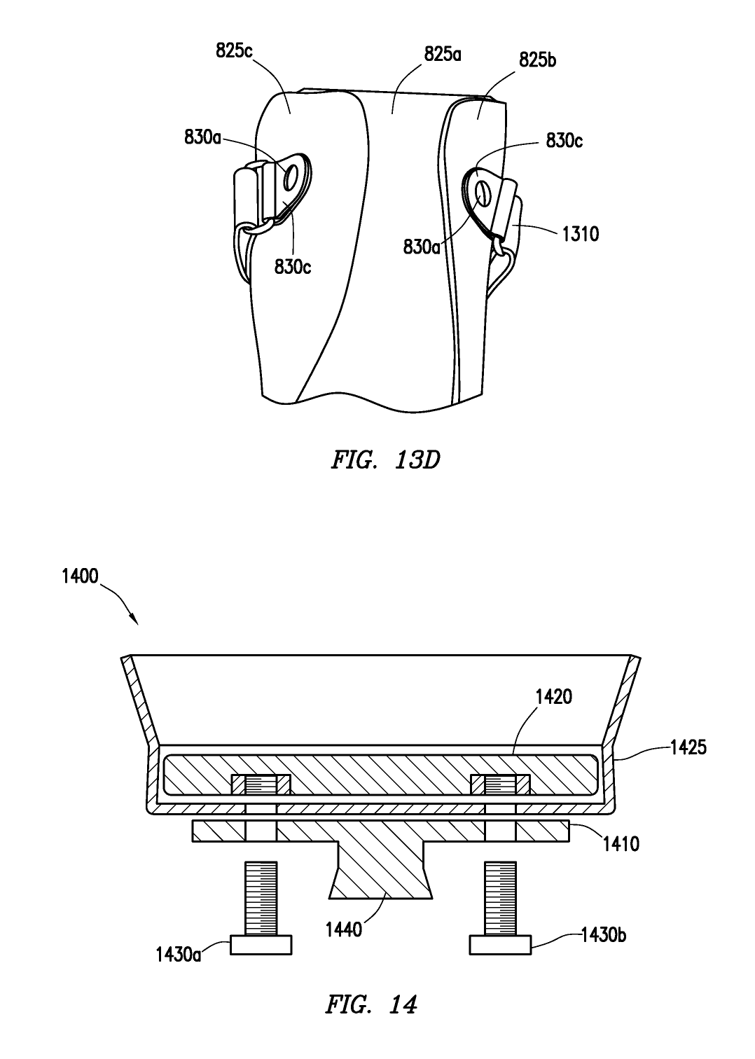

FIGS. 13A-13D illustrate embodiments of various attachment supports 830a-830c, in accordance with the teachings of the present disclosure. In general, attachment supports may facilitate the attachment of a socket to a patient's residual limb. They may also be used to attach other attachment supports to a socket. For example, attachment support 830a may be used to couple attachment support 830c to socket wall 820. In some embodiments, a groove may be placed in socket wall 820 to accommodate attachment support 830a if the socket wall component to which attachment support 830a is coupled to slides or overlaps with another socket wall component such that attachment support 830a must be moved. Alternatively, a groove may be used to facilitate a more comfortable or more secure attachment of a socket to a patient's residual limb. Strap 1310 may be adjustable and used to secure socket wall sections 825 in a configuration consistent with a desired volume. Strap 1310 may be secured with a buckle or any other suitable means of tightening strap 1310.

FIG. 14 illustrates another alternative example embodiment of socket base 1400, in accordance with the teachings of the present disclosure. As shown in FIG. 14, socket base 1400 may include connection plate 1410 comprising engaging member 1440, anchor plate 1420, attachment members 1430a and 1430b, and socket wall components 1425.

The embodiment shown in FIG. 14 is similar to that shown in FIGS. 12A-12C. For example, connection plate 1410 may be similar or identical to connection plate 1210 shown in FIGS. 12A-12C. Attachment members 1430a and 1430b may be similar or identical to attachment members 1230a-1230d shown in FIGS. 12A-12C. However, rather than utilizing base component 1220 that may be configured to engage with a pin coupled to a patient's residual limb, as shown in FIG. 14, anchor plate 1420 may be used to anchor connection plate 1410 to socket base 1400.

In some embodiments, socket wall components 1425 may include channels (not shown) to facilitate rotation of socket wall components 1425 resulting in radial expansion or contraction of socket wall components 1425. These channels may have an arcuate path curving towards the middle of connection plate 1410. In some embodiments, socket wall components 1425 and/or the channels therein may be similar or identical those of the embodiment shown in FIGS. 8-12C.

The internal volume defined by socket wall components 1425 may be modified using the channels, attachment members 1430, and connection plate 1410. For example, by loosening attachment members 1430, socket wall components 1425 may be rotated such that engaging members 1430 remain in the channels. As this occurs, socket wall components 1425 may slide by other socket wall components 1425. Because of the arcuate path of the channels, not only do socket wall components 1425 move around the center of connection plate 1410, but they also move in towards or away from the center of connection plate 1410 as it is moved. By moving socket wall components 1425 along the path defined by the channels and attachment members 1430, and corresponding motion by socket wall components 1425, the volume defined by socket wall components 1425 may be modified. In this way, the volume may be modified in a comparable manner to that observed with respect to the movement of individual wings 215 as shown in FIG. 2 with socket wall components 715 as shown in FIG. 7 coupled thereto. For example, a comparable volume difference of approximately 30% may be observed by moving socket wall components 1425 from one end of the channels to the other.

In some embodiments, supporting members may be placed at various places around the socket to brace or strengthen the socket. For example, a hose clamp may be placed around the bottom of the socket to strengthen the socket. Alternatively, other types of reinforcement or bracing components or members may be used in a variety of places to strengthen the socket.

In some embodiments, design or aesthetic considerations as well as comfort considerations may be taken into account for the design of the socket. For example, the socket base may be sized such that it will fit within a typical pant leg. In some embodiments, an adjustment spacer and/or spacer plate may be sized such that the combination will not extend beyond a traditional socket. In other embodiments, an adjustment spacer may be sized to extend slightly beyond a spacer plate. In some embodiments, there may be a tradeoff between the amount of bulk in the socket base and the amount of radial expansion or contraction that is available. For example, a larger socket base may be used to allow for greater changes in volume, but this may correspond to a larger and/or heavier socket base.

In some embodiments, the weight of various components within a socket and/or socket base may be controlled to prevent the socket from becoming too heavy. For example, in some embodiments the modification from traditional sockets may only increase the weight by approximately 5% or 10%. In some embodiments a socket and/or a socket base may be five pounds or less. In other embodiments, it may be approximately five pounds.

In some embodiments, gel inserts may be placed over some, substantially all, or all of the internal volume of a socket wall. This gel insert may be of varying depths such that minor variations of volume may be inconsequential. This may also facilitate a more comfortable fit for a patient. The gel may be made of any of a variety of gels, including foams, liquid gels beneath a surface layer, or any combinations thereof. The use of gel inserts may also facilitate changes in shape of the residual limb, in addition to changes in volume.

While a variety of examples have been provided and described, it will be appreciated that none of the examples is intended to be limiting. Rather, the examples are provided merely for illustrative purposes to provide assistance in understanding the present disclosure.

Although the present disclosure has been described in detail, it should be understood that various changes, substitutions, and alterations can be made hereto without departing from the spirit and the scope of the disclosure as defined by the appended claims.

1. A radial volume adjustment device comprising:
a spacer plate with one or more channels forming an arcuate path curving towards a center of the spacer plate;
an adjustment spacer consisting of a plurality of sections;
at least one attachment member configured to couple with the spacer plate and pass through the at least one channel such that the at least one adjustment spacer section may move in a path defined by the at least one attachment member and the at least one channel, wherein the movement of the at least one attachment member causes the adjustment spacer sections to move inwardly and outwardly in the radial direction relative to the spacer plate; and
a plurality of socket wall components coupled to the adjustment spacer sections, wherein the adjustment of the adjustment spacer sections changes a volume defined by the plurality of socket wall components.

2. The radial volume adjustment device of claim 1, further including an engaging member configured to couple the spacer plate with an additional prosthetic component.

3. The radial volume adjustment device of claim 1, wherein at least one socket wall component and at least one adjustment spacer section form a unitary body.

4. The radial volume adjustment device of claim 1, wherein one of the plurality of socket wall components is made of rigid material and another of the plurality of socket wall components is made of flexible material.

5. The radial volume adjustment device of claim 1, wherein the plurality of socket wall components are arranged in an alternating pattern between flexible and rigid socket wall components.

6. The radial volume adjustment device of claim 1, wherein the plurality of socket wall components overlap throughout a range of volume adjustment.

7. The radial volume adjustment device of claim 1, further including:
a base component configured to engage with a pin coupled to the residual limb; and
a releasing member configured to release the pin from engagement with the base component.

8. The radial volume adjustment device of claim 1, further including a securing device configured to secure the plurality of socket wall components in a configuration consistent with a volume.

9. The radial volume adjustment device of claim 8, wherein the securing device is an adjustable strap secured with a buckle.

10. A radial volume adjustment device comprising:
a connection plate;
a socket wall comprising a plurality of socket wall components, at least one of the plurality of socket wall components comprising a channel, the channel forming an arcuate path curving towards a center of the connection plate; and
at least one attachment member configured to couple with the connection plate and pass through the at least one channel such that the at least one socket wall component may move in a path defined by the at least one attachment member and the channel, said movement of the at least one attachment member changes a volume defined by the socket wall.

11. The radial volume adjustment device of claim 10, further including an engaging member configured to couple the adjustment spacer with an additional prosthetic component.

12. The radial volume adjustment device of claim 10, further including a securing device configured to secure the plurality of socket wall components in a configuration consistent with a volume.

13. The radial volume adjustment device of claim 12, wherein the securing device is an adjustable strap secured with a buckle.

14. The radial volume adjustment device of claim 10, wherein one of the plurality of socket wall components is made of rigid material and another of the plurality of socket wall components is made of flexible material.

15. The radial volume adjustment device of claim 10, wherein the plurality of socket wall components overlap throughout a range of volume adjustment.

16. A method of manufacturing a radial volume adjustment device comprising:
creating a mold of a residual limb;
crafting a plurality of socket wall components using the mold of the residual limb; inserting a channel in at least one of the plurality of socket wall components, the channel forming an arcuate path curving towards a center of a socket base; and
connecting the plurality of socket wall components to the socket base to form a radial volume adjustment device, wherein the movement of the at least one socket wall component changes a volume defined by the plurality of socket wall components.

17. The method of claim 16, further including attaching an engaging member configured to couple the socket base with an additional prosthetic component.

18. The method of claim 16, wherein one of the plurality of socket wall components is made of rigid material and another of the plurality of socket wall components is made of flexible material.

19. The method of claim 16, wherein connecting the plurality of socket wall components to the socket base further comprises alternating between flexible and rigid socket wall components.

20. The method of claim 16 wherein the plurality of socket wall components overlap throughout a range of volume adjustment.
